(19) United States
(12) Patent Application Publication
(10) Pub. No.: US 2024/0027306 A1
(43) Pub. Date: Jan. 25, 2024

(54) LIQUID SAMPLE SAMPLING DEVICE USED IN OPERATION

(71) Applicant: CHILDREN'S HOSPITAL OF SOOCHOW UNIVERSITY, Suzhou (CN)

(72) Inventors: Feng YAO, Suzhou (CN); Xiaodong WANG, Suzhou (CN); Yunfang ZHEN, Suzhou (CN); Fuyong ZHANG, Suzhou (CN); Xiaofang SHEN, Suzhou (CN); Quanwen YUAN, Suzhou (CN); Zhenhua ZHU, Suzhou (CN); Xianlan XIANG, Suzhou (CN); Xiaolin PAN, Suzhou (CN); Yi QIAO, Suzhou (CN); Wentao YU, Suzhou (CN); Yicong LIU, Suzhou (CN); Zizhen ZHANG, Suzhou (CN); Mengxia LI, Suzhou (CN)

(21) Appl. No.: 18/361,395

(22) Filed: Jul. 28, 2023

Related U.S. Application Data
(63) Continuation of application No. PCT/CN2022/117570, filed on Sep. 7, 2022.

(30) Foreign Application Priority Data
Jan. 13, 2022 (CN) 202210035119.7

(51) Int. Cl. G01N 1/14

(52) U.S. Cl. CPC G01N 1/14; G01N 2001/1418

(57) ABSTRACT

Provided is a liquid sample sampling device used in an operation, and belongs to the technical field of sampling devices. The sampling device includes a negative pressure suction head, an auxiliary tube connected with a side wall of the negative pressure suction head, and a sampling bottle connected with the auxiliary tube. A rebound connecting plate is arranged at a position connecting the negative suction head and the auxiliary tube. The side wall of the negative pressure suction head is provided with a push switch. A diameter of the auxiliary tube is larger than a diameter of the negative pressure suction head. The push switch is an elastic membrane switch.

### CROSS-REFERENCE TO RELATED APPLICATIONS

This disclosure is a continuation of PCT/CN2022/117570, filed on Sep. 7, 2022 and claims priority of Chinese Patent Application No. 202210035119.7, filed on Jan. 13, 2022, the contents of which are hereby incorporated by reference.

### TECHNICAL FIELD

The disclosure relates to the technical field of sampling devices, and in particular to a liquid sample sampling device used in an operation.

### BACKGROUND

When there is a need to sample a liquid sample during an operation, a disposable syringe (divided into a needle and a syringe) is usually used for aspiration. The liquid sample sampled by the disposable syringe is usually sent for inspection together with the syringe after sampling, or transferred to a sample delivery tube or an other temporary storage or inspection device after the sampling, followed by further operations such as culture, inspection and freezing. However, when the syringe is used for the aspiration, puncture with a syringe needle is easy to cause damage to adjacent and deep tissues, and an operation of aspiration sampling is difficult. Moreover, a sample is difficult to store in a sealed manner after the sampling is completed, and is prone to leakage at the needle or a syringe head. Therefore, the disposable syringe has many shortcomings.

In the prior art, when sampling the liquid sample, following technical means are often used for the sampling.

1. Aspiration sampling through a syringe (needle, syringe) is most commonly used.

2. Sampling with tweezers is not feasible for a liquid tissue sample.

3. Sampling through complete removal of a cyst during an operation is difficult to operate and may cause significant damage to a patient.

4. When sampling through negative pressure suction, a conventional negative pressure aspirator used during an operation may extract a sample, but may not sample a target sample (the sample may only be sucked into a waste liquid tank and discarded).

5. Collecting and sampling through a three-way aspirator combined with a syringe is difficult to collect a sample from an abdomen and pelvic cavity with a small amount. Poor sealing of connection at a joint between the syringe and the aspirator often leads to a leakage of sample liquid, resulting in environmental pollution in an operating room. If a patient is infected with some infectious pathogens, safety of an operator may be directly threaten.

6. It is difficult to take a liquid sample from an abdomen and pelvic cavity and collect a sample with a small amount by using a sample bag (taking appendix and other samples). Moreover, when collecting a sample with an enough amount, the sample is easy to leak out and cause pollution.

7. The prior art also discloses a device (Dongtai People's Hospital, Surgical Fluid and Sample Recovery Device: CN202021757989.82021-05-25.), which may effectively solve a problem of pollution to an operator with washing fluid flowing out during a minimal invasive operation such as an electrosurgical excision procedure and a laser lithotripsy surgery and recovery of resect tissue samples, ensure that sterile areas related to a surgeon and an operating room are not polluted by the washing fluid, and effectively avoid loss and contamination of the sample. The surgical liquid and sample recovery device includes a film pasting sheet and a recovery part. The recovery part includes an iron wire ring, a liquid collecting bag and a liquid collecting tube. The liquid collecting bag includes an upper opening and a lower opening. One part of an edge of the upper opening is connected with the film pasting sheet and an other part is connected with the iron wire ring. The lower opening of the liquid collecting bag is connected with the liquid collecting tube, and a plurality of leakage holes are arranged at a lower part of the liquid collecting tube. This device may collect the liquid drained out of a body, but it may not complete a process of collecting the liquid in the body.

### SUMMARY

In view of this, in order to solve a technical problem that there is no device, instrument or tool specially used for sampling a liquid sample in an operation in the prior art, the disclosure provides a liquid sample sampling device used in an operation, which may realize more convenient and accurate sampling and inspection of the liquid sample in the operation, and avoid a leakage of the liquid sample to pollute surrounding tissues and to cause an iatrogenic injury during or after sampling.

In order to achieve an above objective, the disclosure provides a following technical scheme.

The liquid sample sampling device used in the operation includes a negative pressure suction head, an auxiliary tube connected with a side wall of the negative pressure suction head, and a sampling bottle connected with the auxiliary tube.

A rebound connecting plate for communicating and closing the negative pressure suction head is arranged at a position connecting the negative suction head and the auxiliary tube.

The side wall of the negative pressure suction head is provided with a push switch capable of opening and closing the rebound connecting plate.

A diameter of the auxiliary tube is larger than a diameter of the negative pressure suction head.

The push switch is an elastic membrane switch.

When the elastic membrane switch is pressed, a soft membrane on the elastic membrane switch moves in a direction of the rebound connecting plate, and is used to block a flow of liquid and drive the rebound connecting plate to open, making the auxiliary tube communicated with the negative pressure suction head.

When the elastic membrane switch is released, the soft membrane returns to an original position, the liquid flows in the negative pressure suction head, the rebound connecting plate returns to an original position, and the auxiliary tube is not communicated with the negative pressure suction head.

Optionally, the soft membrane is provided with a protrusion.

Optionally, a sampling part is a circular tube.

Compared with the prior art, the disclosure has following beneficial effects.

Firstly, the liquid sample sampling device used in the operation provided by the disclosure may realize accurate sampling, prevent the sample from leaking, save an operation time, send for inspection, etc., and avoid a leakage of the liquid sample to pollute the surrounding tissues and cause the iatrogenic injury during or after the sampling.

Secondly, the liquid sample sampling device used in the operation provided by the disclosure may realize more convenient sampling, and make a sampled sample more complete.

The liquid sample may be prevented from leaking, and interfering components (such as peripheral blood seepage, etc.) in the sample are reduced, and mixing is minimized.

A sampling opportunity may be controlled, the sampling is accurate, and a sampling time may be saved and shortened.

The liquid sample sampling device used in the operation provided by the disclosure may be perfectly combined with a negative pressure suction system used in the operation, and a complete and accurate suction sample is sucked by negative pressure, so that the sample is convenient to transfer, sample pollution is avoided, sample pollution to a surrounding operation area caused by untimely sampling is avoided, and the iatrogenic injury such as pollution to the surrounding operation area caused by a sample leakage is prevented.

Thirdly, compared with conventional syringe sampling, the liquid sample sampling device used in the operation provided by the disclosure is more convenient, saves the operation time, carries out sampling more timely, is easier to operate, avoids the sample leakage, and is perfectly combined with a negative pressure system in the operation. The liquid sample is sampled more accurately, and the pollution of a surrounding environment to the sample is avoided.

### BRIEF DESCRIPTION OF THE DRAWINGS

FIG. 1 is a schematic structural view of a push switch in a closed state according to the disclosure.

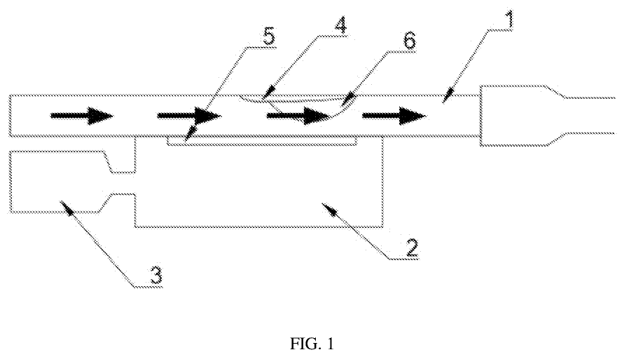

FIG. 2 is a schematic structural view of a push switch in an open state according to the disclosure.

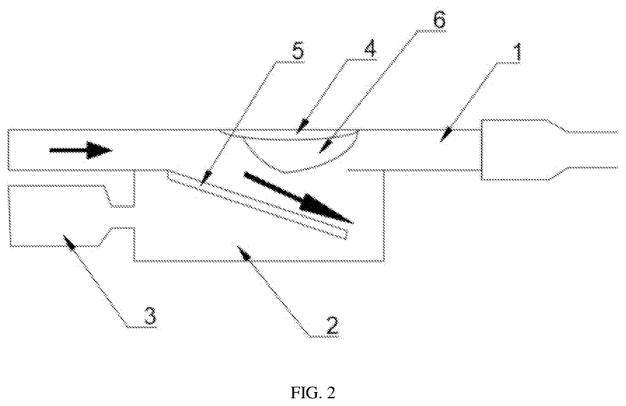

### DETAILED DESCRIPTION OF THE EMBODIMENTS

In the following, technical schemes in embodiments of the disclosure may be clearly and completely described with reference to attached drawings. Obviously, the described embodiments are only a part of the embodiments of the disclosure, but not all embodiments. Based on the embodiments in the disclosure, all other embodiments obtained by ordinary technicians in the field without a creative labor belong to a scope of protection of the disclosure.

In a description of the disclosure, it should be noted that azimuth or positional relationships indicated by terms “upper”, “lower”, “inner”, “outer” and “top/bottom” are based on azimuth or positional relationships shown in the attached drawings, and are only for the convenience of describing the disclosure and simplifying the description, and does not indicate or imply that a referred device or an element must have a specific orientation, be constructed and operated in a specific orientation, therefore it may not be understood as a limitation of the disclosure. In addition, terms “first” and “second” are only used for descriptive purposes and may not be understood as indicating or implying relative importance.

In the description of the disclosure, it should be noted that unless otherwise specified and limited, terms “installed”, “provided”, “sheathed/sleeved” and “connected” should be broadly understood. For example, “connected” may be fixedly connected, detachably connected or integrated, may be mechanically connected or electrically connected, may be directly connected, may also be indirectly connected through an intermediate medium, and may be connected inside two elements. For those skilled in the art, specific meanings of the above terms in the disclosure may be understood in specific situations.

As shown in FIG. 12, the disclosure provides a liquid sample sampling device used in an operation, including a negative pressure suction head 1, an auxiliary tube 2 connected with a side wall of the negative pressure suction head 1, and a sampling bottle 3 connected with the auxiliary tube 2.

A rebound connecting plate 5 for communicating and closing the negative pressure suction head 1 is arranged at a position connecting the negative suction head 1 and the auxiliary tube 2.

The side wall of the negative pressure suction head 1 is provided with a push switch capable of opening and closing the rebound connecting plate 5.

A diameter of the auxiliary tube 2 is larger than a diameter of the negative pressure suction head 1.

The push switch is an elastic membrane switch 4.

When the elastic membrane switch 4 is pressed, a soft membrane on the elastic membrane switch 4 moves in a direction of the rebound connecting plate 5, and is used to block a flow of liquid and drive the rebound connecting plate 5 to open, making the auxiliary tube 2 communicated with the negative pressure suction head 1.

When the elastic membrane switch 4 is released, the soft membrane returns to an original position, the liquid flows in the negative pressure suction head 1, the rebound connecting plate 5 returns to an original position, and the auxiliary tube 2 is not communicated with the negative pressure suction head 1.

In the disclosure, the soft membrane is provided with a protrusion 6.

In the disclosure, a sampling part is a circular tube.

In the sampling device provided by the disclosure, the elastic membrane switch 4 may be a section of soft membrane arranged on a tube wall of the negative pressure suction head 1, and the soft membrane has elasticity. When the soft membrane is pressed, the flexible membrane moves in the direction of the rebound connecting plate 5, so that the flexible membrane contacts the rebound connecting plate 5, and then the rebound connecting plate 5 moves in the auxiliary tube 2 to make the auxiliary tube 2 communicate with the negative pressure suction head 1.

The soft membrane has a blocking effect on the liquid flowing in the negative pressure suction head 1 to block a linear movement of the liquid attracted by the negative pressure suction head 1, so that the attracted liquid changes its moving direction in a section of the auxiliary tube. At this time, the liquid flows into the auxiliary tube 2. Since the diameter of the auxiliary tube 2 is larger than the diameter of the negative pressure suction head 1, a flow rate may be slowed down because a cross-sectional area becomes larger at this time. When the flow rate is reduced enough, gas and the liquid may be separated due to gravity and inertia. A liquid sample separated from the sampling bottle 3 is used for related tests, cultures, etc.

The soft membrane on the elastic membrane switch 4 may also be provided with the protrusion 6, which is more convenient to use. Among them, the rebound connecting plate 5 may be made of rubber or latex, so the rebound connecting plate 5 has certain elasticity and may automatically rebound, making the auxiliary tube 2 and the negative pressure suction head 1 closed and not communicated.

The above is only a preferred embodiment of the disclosure. However, a protection scope of the disclosure is not limited to this. Any equivalent substitution or change made by any person skilled in the art, within a technology scope disclosed herein, in accordance with the technical schemes of the disclosure and its improved conception, shall be covered by the protection scope of the disclosure.

What is claimed is:

1. A liquid sample sampling device used in an operation, comprising a negative pressure suction head, an auxiliary tube connected with a side wall of the negative pressure suction head, and a sampling bottle connected with the auxiliary tube;
wherein a rebound connecting plate for communicating and closing the negative pressure suction head is arranged at a position connecting the negative pressure suction head and the auxiliary tube;
the side wall of the negative pressure suction head is provided with a push switch capable of opening and closing the rebound connecting plate;
a diameter of the auxiliary tube is larger than a diameter of the negative pressure suction head;
the push switch is an elastic membrane switch;
when the elastic membrane switch is pressed, a soft membrane on the elastic membrane switch moves in a direction of the rebound connecting plate, and is used to block a flow of liquid and drive the rebound connecting plate to open, making the auxiliary tube communicated with the negative pressure suction head; and
when the elastic membrane switch is released, the soft membrane returns to an original position, the liquid flows in the negative pressure suction head, the rebound connecting plate returns to an original position, and the auxiliary tube is not communicated with the negative pressure suction head.

2. The liquid sample sampling device used in the operation according to claim 1, wherein the soft membrane is provided with a protrusion.
